(19) United States
(12) Patent Application Publication
(10) Pub. No.: US 2022/0062464 A1
(43) Pub. Date: Mar. 3, 2022

(54) APPARATUS FOR CONTAINMENT OF PATHOGENS

(71) Applicant: John Lyng, Minnetonka, MN (US)

(72) Inventor: John Lyng, Minnetonka, MN (US)

(21) Appl. No.: 17/009,442

(22) Filed: Sep. 1, 2020

(51) Int. Cl. A61L 2/10

(52) U.S. Cl. CPC A61L 2/10; A61L 2202/11; A61L 2202/20; A61L 2202/122; A61L 2202/15; A61L 2202/16

(57) ABSTRACT

The pathogen containment apparatus disclosed herein is alterable between a deployed position and a storage position. In the deployed position, a hood defines a hood chamber that partially encloses a head and upper torso of a patient. Air that may include pathogens from the patient or the ambient environment is communicated sequentially from the ambient environment through the hood chamber, through a duct, and through a container chamber. A filter and a UV lamp may remove pathogens from the air as the air is being communicated. The pathogen containment apparatus may be altered from the deployed position into the storage position wherein the pathogen containment apparatus is condensed into the container chamber of the container to allow transport or storage.

### BACKGROUND OF THE INVENTION

### Field

The present disclosure relates to medical equipment, and, more particularly, to portable medical equipment for the containment of infected patients having an airborne or droplet communicable disease.

### Background

COVID-19, which is caused by a virulent coronavirus, has proven to be a highly communicable disease that may have a mortality rate higher than that of seasonal influenza. While elderly patients with underlying medical conditions are most at risk, everyone is vulnerable. COVID-19 patients pose a disease communication risk to everyone in a healthcare setting including healthcare providers such as doctors, nurses, and other medical personnel as well as other patients.

Because COVID-19 is spreading rapidly through the population, a large number of COVID-19 patients may require treatment in a healthcare facility at a given time. However, this large number of patients may exceed available resources. While healthcare facilities typically have various protective equipment such as isolation rooms, isolation wards, tenting, and various other equipment that may be used to contain the spread of COVID-19 from those infected, the demand may exceed the availability of such resources. Furthermore, it may be necessary to treat COVID-19 patients in other setting such as field hospitals, ships, arenas, commercial buildings, and other such facilities that may not be equipped to contain the transmission of highly communicable disease. Protection is also required during patent transport such a patent transport in ambulances and in air ambulances whether helicopters or fixed wing aircraft. Lack of sufficient protective equipment to contain the spread of COVID-19 endangers healthcare providers and non-COVID patients.

Moreover, COVID-19 is but one example of a highly communicable disease. There have been various recent outbreaks of other dangerous and highly communicable airborne diseases such as, for example, SARS, influenza (e.g., Spanish flu, Asian flu, Hong Kong flu), Marburg virus disease, Ebola, meningitis, bubonic plague, and measles. It is quite possible that patients having such diseases could overwhelm available healthcare resources. In addition, it may be necessary to provide treatment to patients having such diseases who are located in remote or underdeveloped areas that lack the protective equipment required to contain the spread of such dangerous and highly communicable diseases.

Accordingly, there is a need for improved apparatus as well as related methods that contain the spread of communicable diseases transmissible via airborne and droplet routes from infected patients.

### BRIEF SUMMARY OF THE INVENTION

These and other needs and disadvantages may be overcome by the apparatus and related methods of use disclosed herein. Additional improvements and advantages may be recognized by those of ordinary skill in the art upon study of the present disclosure.

In various aspects, the pathogen containment apparatus disclosed herein includes a container that defines a container chamber within, and a hood alterable between a hood storage position and a hood deployed position. The hood in the hood deployed position defines a hood chamber to partially enclose a head and upper torso of a patient, in various aspects. In various aspects, the pathogen containment apparatus disclosed herein includes a duct alterable between a duct storage position and a duct deployed position. The duct in the duct deployed position is operably connectable to the hood in the hood deployed position and to the container to define a duct passage that forms at least a portion of a fluid pathway along which air is communicated into the hood chamber from an ambient environment, from the hood chamber to the container chamber, and from the container chamber through a vent to the ambient environment, in various aspects. The hood in the hood storage position and the duct in the duct storage position may be enclosed within the container chamber of the container.

In various aspects, the pathogen containment apparatus includes a filter positionable within the fluid pathway to remove pathogens from air communicated via the fluid pathway, a lamp positionable within the fluid pathway that emits an ultraviolet-c (UVC) wavelength light to disinfect air communicated via the fluid pathway, and a reflective material that forms an interior surface enclosing portions of the fluid pathway to reflect the ultraviolet-c (UVC) wavelength light within the fluid pathway in order to enhance disinfection.

This summary is presented to provide a basic understanding of some aspects of the apparatus and methods disclosed herein as a prelude to the detailed description that follows below. Accordingly, this summary is not intended to identify key elements of the apparatus and methods disclosed herein or to delineate the scope thereof.

### BRIEF DESCRIPTION OF THE DRAWINGS

FIG. 1A illustrates by perspective view an exemplary implementation of a pathogen containment apparatus in a deployed state;

FIG. 1B illustrates by perspective view portions of the exemplary pathogen containment apparatus of FIG. 1A;

FIG. 2A illustrates by perspective view portions of the exemplary pathogen containment apparatus of FIG. 1A in the deployed state;

FIG. 2B illustrates by perspective view portions of the exemplary pathogen containment apparatus of FIG. 1A in a storage state;

FIG. 2C illustrates by perspective view other portions of the exemplary pathogen containment apparatus of FIG. 1A in the storage state;

FIG. 2D illustrates by cut-away plan view portions of the exemplary pathogen containment apparatus of FIG. 1A in the deployed state;

FIG. 3 illustrates by plan view portions of the exemplary pathogen containment apparatus of FIG. 1A in the deployed state;

FIG. 4 illustrates by schematic view portions of the exemplary pathogen containment apparatus of FIG. 1A in the storage state;

FIG. 5 illustrates by schematic diagram portions of the exemplary pathogen containment apparatus of FIG. 1A; and,

FIG. 6 illustrates by process flow chart an exemplary method of operation of the pathogen containment apparatus of FIG. 1A including altering the pathogen containment apparatus between the storage state and the deployed state.

The Figures are exemplary only, and the implementations illustrated therein are selected to facilitate explanation. The number, position, relationship and dimensions of the elements shown in the Figures to form the various implementations described herein, as well as dimensions and dimensional proportions to conform to specific force, weight, strength, flow and similar requirements are explained herein or are understandable to a person of ordinary skill in the art upon study of this disclosure. Where used in the various Figures, the same numerals designate the same or similar elements. Furthermore, when the terms “top,” “bottom,” “right,” “left,” “forward,” “rear,” “first,” “second,” “inside,” “outside,” and similar terms are used, the terms should be understood in reference to the orientation of the implementations shown in the drawings and are utilized to facilitate description thereof. The steps in the various methods illustrated in the Figures, for example, may be performed in other orders, or the steps in the various methods may be divided or subdivided in various ways, in other implementations. Methods, in other implementations, may include steps additional to those illustrated or may not include certain steps of the illustrated methods. Use herein of relative terms such as generally, about, approximately, essentially, may be indicative of engineering, manufacturing, or scientific tolerances such as ±0.1%, ±1%, ±2.5%, ±5%, or other such tolerances, as would be recognized by those of ordinary skill in the art upon study of this disclosure.

### DETAILED DESCRIPTION OF THE INVENTION

The pathogen containment apparatus disclosed herein, in various aspects, includes a container that defines a container chamber within, and includes a hood that forms a hood chamber that partially encloses the head and upper torso of a patent. A duct fluidly connects the hood chamber with the container chamber, and a fan unit disposed within the container chamber communicates air along a fluid pathway from the ambient environment into the hood chamber, from the hood chamber through the duct into the container chamber, and discharges the air from the container chamber through a vent back into the ambient environment. The air may be filtered by a filter to remove pathogens and disinfected by a lamp that emits UV light as the air is communicated along the fluid pathway between the hood chamber and the vent so that air generally devoid of pathogens is discharged from the vent. Accordingly, the pathogen containment apparatus contains pathogens emitted by the patient as well as pathogens in the ambient environment by filtration and/or destruction of said pathogens. A reflective material may be disposed about portions of the fluid pathway between the hood chamber and the vent to reflect the UV light in order to enhance disinfection by the UV light.

The pathogen containment apparatus may be operable between a storage state and a deployed state. In the storage state, the hood positioned in a hood storage state, the duct positioned in a duct storage state, and other components of the pathogen containment apparatus may be placed within the container chamber thereby consolidating the pathogen containment apparatus into the container. The pathogen containment apparatus in the storage state may then be transported or stored by transporting or storing the container with the hood, duct, and other components disposed therein.

In the deployed state, the hood in a hood deployed state defining a hood chamber is in fluid communication with the container chamber by the duct in a duct deployed state, in various aspects. In the deployed state, the pathogen containment apparatus is operable to communicate air from the hood chamber, disinfect the air by filtration, by UV light, or by both filtration and UV light, and then discharge the disinfected air into the ambient environment, in various aspects.

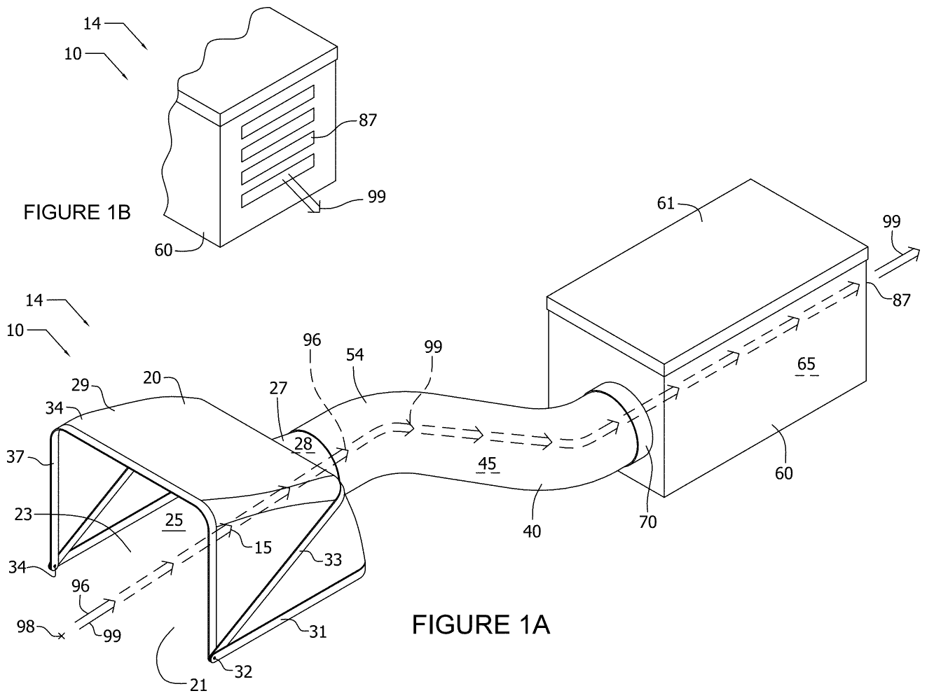

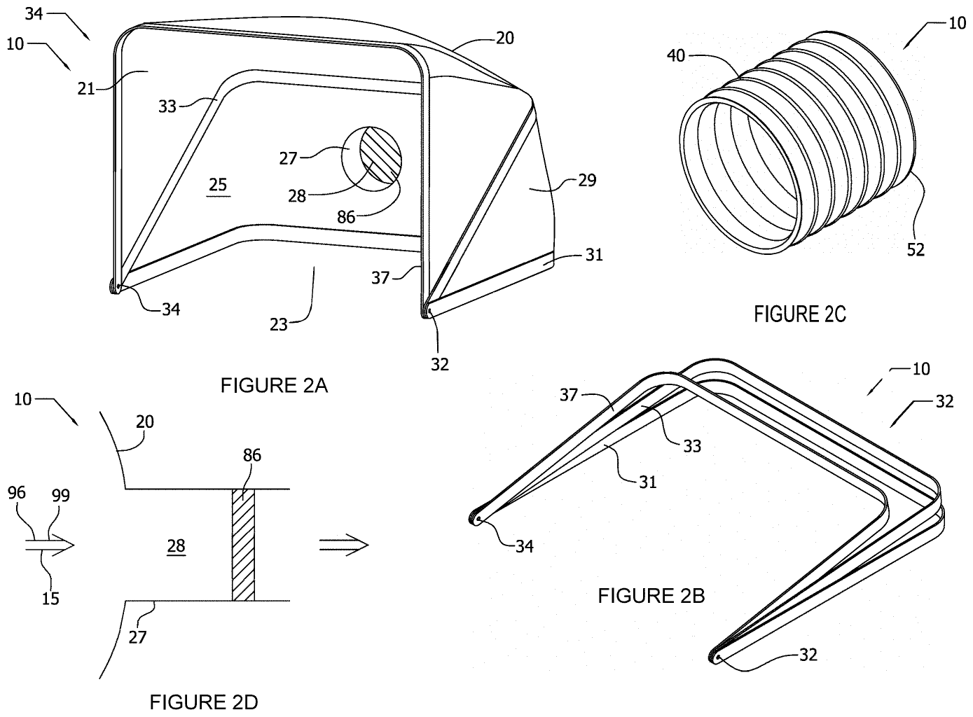

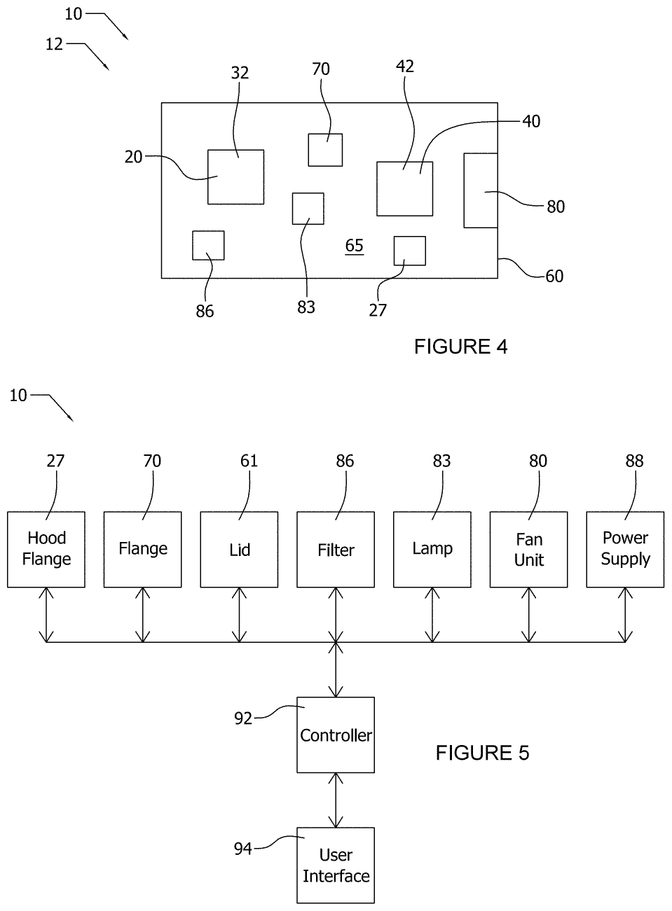

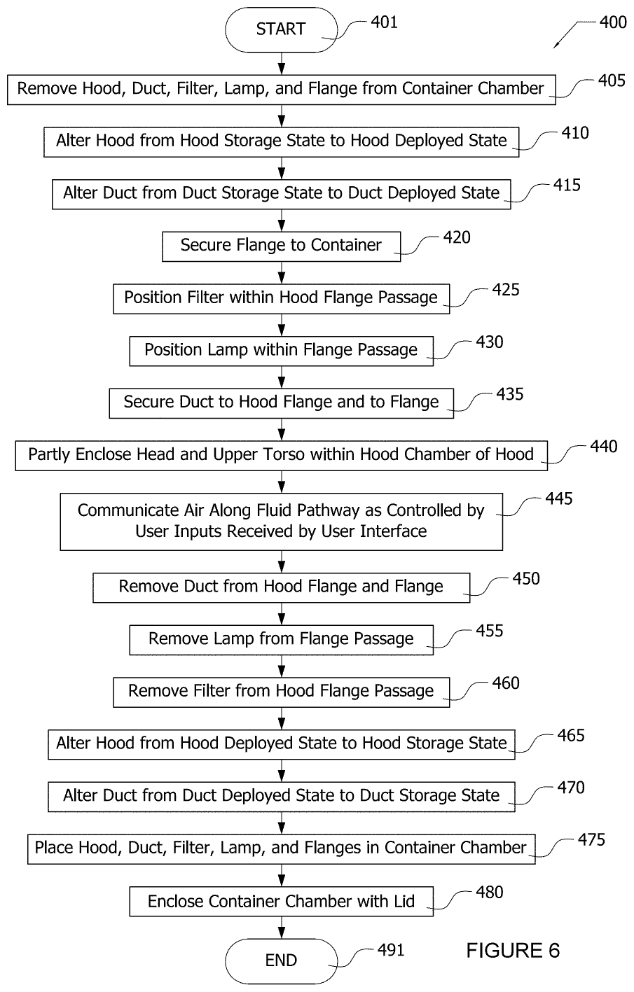

As illustrated in FIGS. 1A, 1B, exemplary pathogen containment apparatus 10 includes hood 20 that may fluidly communicate with container 60 by duct 40. Hood 20 may be altered between hood storage state 32 (see FIG. 2B) and hood deployed state 34, and duct 40 may be altered between duct storage state 52 (see FIG. 2C) and duct deployed state 54 thereby allowing pathogen containment apparatus 10 to be altered between storage state 12 (see FIG. 4) and deployed state 14. Pathogen containment apparatus 10 is illustrated in deployed state 14 with hood 20 in hood deployed state 34 and duct 40 in duct deployed state 54 in FIGS. 1A, 1B.

Hood 20 in hood deployed state 34 may be placed over a head and an upper torso of a patent with the head and upper torso inserted into or otherwise positioned within hood aperture 23 (also see FIG. 2A) thereby defining hood chamber 25 around the head and the upper torso of the patient that partially encloses the head and the upper torso. Chamber 25 is open to the ambient environment 98 through aperture 21 so that the head and upper torso are not entirely enclosed within chamber 25, in this implementation. The patient may be lying in a prone, lateral decubitus, supine, or semi-recumbent position upon a bed including, for example, a litter, a cot, a bed, and a stretcher. Various straps including other fittings may be disposed about hood 20 to secure hood 20 in deployed state 34 about the patient, in various implementations.

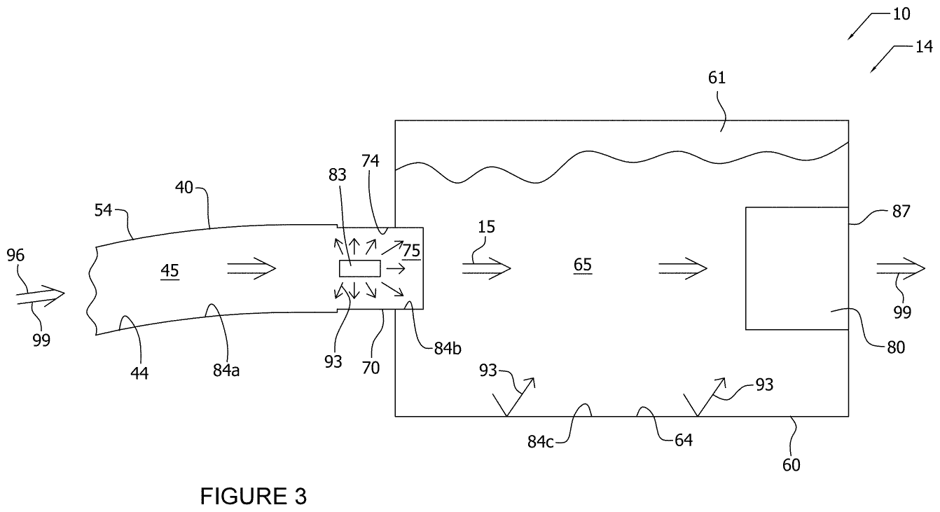

Hood flange 27, which is secured to hood 20, is placed proximate a crown of the head, in this exemplary implementation. Duct 40 in duct deployed state 54 is attached to hood flange 27 and to flange 70 to communicate air 99 from hood chamber 25 into container chamber 65 of container 60 in part through duct passage 45 of duct 40, in this implementation. Thus, as illustrated in FIGS. 1A, 1B, air 99 is communicated along fluid pathway 15 from the ambient environment 98 through hood aperture 21 into hood chamber 25, from hood chamber 25 through hood flange passage 28 of hood flange 27 into duct passage 45 of duct 40, from duct passage 45 through flange passage 75 of flange 70 (see FIG. 3) into container chamber 65 of container 60, and from container chamber 65 into the ambient environment 98 through vent 87. Accordingly, in this implementation, fluid pathway 15 includes ambient environment 98, hood aperture 21, hood chamber 25, hood flange passage 28 of hood flange 27, duct passage 45 of duct 40, flange passage 75 of flange 70, container chamber 65 of container 60, and vent 87.

Vent 87 is located on container 60 opposite of flange 70 so that air 99 is communicated generally through an entirety of container chamber 65 for discharge through vent 87 into ambient environment 98, as illustrated. Air 99 entrains pathogens 96 emitted from the patient as well as pathogens from ambient environment 98 as air 99 is communicated from hood chamber 25 into duct passage 45, in this implementation. Pathogens, as used herein, may include fungi, bacteria, viruses, protozoa, other disease-causing organisms, as well as bodily fluids such as liquid droplets and aerosols, and various noxious, odiferous, or undesirable substances as may be entrained in air 99. In this implementation, pathogens 96 are removed from air 99 as air 99 is communicated along fluid pathway 15, for example, by filter 86 (see FIG. 2D) and by UV light 93 (see FIG. 3) so that air 99 generally devoid of pathogens 96 is discharged into ambient environment 98 through vent 87.

FIGS. 2A, 2B illustrate hood 20 in hood deployed state 34 (also see FIG. 1A) and in hood storage state 32, respectively. As illustrated, hood 20 includes sheeting 29 secured to struts 31, 33, 37 with struts 31, 33, 37 being pivotably connected to one another by pivots 32, 34. Sheeting 29 may be secured to struts 31, 33, 37, for example, by mechanical fasteners, insertion of struts 31, 33, 37 into pockets formed in sheeting 29, forming sheeting 29 around struts 31, 33, 37, or in other ways, as would be readily recognized by those of ordinary skill in the art upon study of this disclosure. In hood deployed state 34 illustrated in FIGS. 1A, 2A, struts 31, 33, 37 are pivoted to tension sheeting 29 therebetween thereby defining hood chamber 25 with hood apertures 21, 23. Hood aperture 23 may be placed over the head and the upper torso of the patient so that hood chamber 25 partly encloses the head and the upper torso. With hood 20 in hood deployed state 34, pivots 32, 34 may be locked to hold struts 31, 33, 37 in fixed relation with one another thereby maintaining hood 20 in hood deployed state 34, in certain implementations. In other implementations, pivots 32, 34 may frictionally engage struts 31, 33, 37 with one another thereby frictionally holding struts 31, 33, 37 in fixed relation with one another thus maintaining hood 20 in hood deployed state 34. Sheeting 29 may be formed of flexible transparent plastic sheeting. Struts 31, 33, 35 may be formed of plastic or metal such as aluminum, and hood flange 27 may be formed of plastic, metal, or combinations thereof. Hood flange 27 may be permanently or removably attached to sheeting 29, in various implementations. In implementations in which hood flange 27 is removable, hood flange 27 is attached to sheeting 29 in hood deployed state 34. With the hood in hood deployed state 34, a caregiver may access the patient, for example, through hood aperture 21 as hood 20 protects the caregiver.

FIG. 2B illustrates hood 20 in hood storage state 32, for example, for transport or storage of hood 20. In hood storage state 32, struts 31, 33, 37 are pivoted with respect to one another to collapse sheeting 29 (note sheeting 29 is omitted from FIG. 2B for purposes of clarity of depiction). Struts 31, 33, 37 may nest within one another in hood storage state 32, and sheeting 29 may be furled around struts 31, 33, 37. Pivots 32, 34 may be locked to hold struts 31, 33, 37 in fixed relation with one another thereby maintaining hood 20 in hood storage state 32. For example, pivots 32, 34 may be released to allow hood 20 to be altered between hood storage state 32 and hood deployed state 34 pivoting of struts 31, 33, 37. In implementations in which hood flange 27 is removable, hood flange 27 is removed from sheeting 29 in hood storage state 32.

FIG. 2C illustrates duct 40 in duct storage state 52. Duct 40 may be, for example, a 6-inch diameter flexible HVAC ducting that may be compressed from duct deployed state 54 into duct storage state 52 and expanded from duct storage state 52 into duct deployed state 54, as would be readily recognized by those of ordinary skill in the art upon study of this disclosure. Duct 40 in duct deployed state 54 attaches to hood flange 27 of hood 20 and flange 70 of container 60 by clamp, frictional engagement, or suchlike, as would be readily recognized by those of ordinary skill in the art upon study of this disclosure. Duct 40 may be comprised of, for example, various combinations of plastic and aluminum. Duct 40, for example, may be a three-layer HVAC flexible ducting with the interior most layer comprised of aluminum for reflectivity.

FIG. 2D illustrates filter 86 disposed within hood flange passage 28 of hood flange 27, as illustrated. Filter 86 may be, for example, a high efficiency particulate air (HEPA) filter. Filter 86 may remove at least 99.97% of airborne particles 0.3 μm in diameter in implementations conforming to U.S. Dept. of Energy standards. Filter 86 in other implementations may, for example, conform to European Standard EN 1822-1:2009.

FIG. 3 illustrates container 60 including container chamber 65. Lid 61 is engaged with the remainder of container 60 to enclose sealingly container chamber 65. Lid 61 may be, for example, hingedly or removably frictionally engaged with the remainder of container 60 to allow access to container chamber 65. A gasket (not shown), for example, may be included between lid 61 and the remainder of container 60 to sealingly enclose container chamber 65 with lid 61. Fan unit 80 is disposed within container chamber 65 to communicate air 99 along fluid pathway 15 from the ambient environment 98 through hood aperture 21 into hood chamber 25, from hood chamber 25 through hood flange passage 28 of hood flange 27 into duct passage 45 of duct 40, and from duct passage 45 through flange passage 75 of flange 70 into container chamber 65 of container 60. Fan unit 80, which is proximate vent 87, discharges air 99 from container chamber 65 into the ambient environment 98 through vent 87.

Duct 40 in duct deployed state 54 is received onto flange 70 that extends forth from container 60, as illustrated, so that duct passage 45 of duct 40 fluidly communicates with flange passage 75 of flange 70 that, in turn, fluidly communicates with container chamber 65 of container 60. Lamp 83 is disposed within flange passage 75, as illustrated. For example, lamp 83 may be engaged with a keyless E26-base lamp socket disposed within flange passage 75. In various implementations, a switch (not shown) is installed on container 60 that is activated to allow power to flow onto lamp 83 by proper securement of lid 61 to the remainder of container 60. The switch prevents operation of lamp 83 unless lid 61 is properly secured to enclose sealingly container chamber 65 thereby preventing human exposure to UV light 93.

Interior surface 44 of duct 40, interior surface 74 of flange 70, and interior surface 64 of container 60, are formed of materials 84a, 84b, 84c, respectively, that reflect UV light 93 emitted by lamp 83, and lamp 83 may be positioned to emit UV light 93 that reflects off of at least portions of interior surfaces 44, 64, 74. Lamp 83 emits UV light 93 (indicated by arrows in FIG. 3) that may comprise at least portions of the UVC spectrum (280 nm-100 nm wavelength). In certain implementations, lamp 83 emits UV light 93 comprising wavelengths between about 270 nm and about 250 nm. Lamp 83 may be, for example, a UV-C light emitting diode (LED) or a mercury vapor lamp that emits UV light 93 at least in the UV-C spectrum. Because vent 87 is located on container 60 opposite of flange 70, air 99 is communicated generally through a length of container chamber 65 between flange 70 and vent 87 to increase exposure of pathogens 98 in air 99 to UV light 93 reflected from interior surface 64 of container 60.

FIG. 4 illustrates pathogen containment apparatus 10 in storage state 12 with hood 20 in hood storage state 32 and duct 40 in storage state 42 disposed within container chamber 65 of container 60. In implementations in which hood flange 27 is removable, hood flange 27, which is removed from sheeting 29, is disposed within container chamber 65 of container 60 separate from hood 20 in storage state 32. Hood flange 27 is illustrated as separated from a remainder of hood 20 in FIG. 4. Lid 61 may be secured onto container 60 to enclose container chamber 65 in storage state 12. As illustrated in FIG. 4, flange 70 may be removed from container 60 and then disposed within container chamber 65 along with lamp 83 in storage state 12. In other implementations, flange 70 may be permanently affixed to container 60, and flange 70 may be repositioned in various ways so as to not extend forth from container, in storage state 12. Filter 86 may be removed from hood flange 27 and disposed within container chamber 65. Container 60 including lid 61 may be constructed of various plastics such as high-density polyethylene (HDPE), polypropylene (PP), polyvinyl chloride (PVC), and acrylonitrile butadiene styrene (ABS). Container 60 may be rectangular and sized, for example, 32 in×18 in×20 in (length×width×height) to allow 5 containers 60 to fit into a Western Shelter Storage Case: CA-94342ITC 94×34×21.

As illustrated in FIG. 5, exemplary pathogen containment apparatus 10 includes controller 92 operably connected to hood flange 27, flange 70, lid 61, fan unit 80, lamp 83, filter 86, power supply 88, and user interface 94. Controller 92 controls the flow of electrical power onto fan unit 80 from power supply 88 to regulate operations of fan unit 80, in this implementation. Power supply 88 may be mains electric, battery, or a combination of mains electric and battery, in various implementations. User interface 94, for example, communicates information to the user that is received from the controller 92 indicative of operations of pathogen containment apparatus 10. User interface 94 communicates user inputs received from the user to the controller 92, for example, that control operations of pathogen containment apparatus 10. User interface 94 may include various visual and audio devices for the communication of information to the user and for the receipt of user inputs from the user, as would be readily recognized by those of ordinary skill in the art upon study of this disclosure. Controller 92 may include a microcontroller, various analog switches and other digital devices, analog devices, sensors, and so forth, as would be readily recognized by those of ordinary skill in the art upon study of this disclosure.

Controller 92 may implement various safety interlocks, for example, that prevent operation of pathogen containment apparatus 10 unless lid 61 is properly secured to the remainder of container 60, filter 86 is properly positioned within hood flange passage 28 of hood flange 27, and duct 40 is properly engaged with hood flange 27 and with flange 70. User interface 94 may provide an audible warning and/or a visual warning indicative of a fault condition such as, for example, improper securement of lid 61, improper positioning of filter 86, failure of lamp 83, and improper engagement of duct 40 with flange 70 or with hood flange 27.

Although omitted for purposes of clarity of explanation, it should be understood that pathogen containment apparatus 10, in various implementations, may include various electrical pathways, data communication pathways, sensor(s), digital communication interface(s), lighting, and so forth, as would be readily recognized by those of ordinary skill in the art upon study of this disclosure.

In operation, pathogen containment apparatus 10 may be initially in storage state 12 with hood 20 in hood storage state 32, duct 40 in duct storage state 52, hood flange 27, flange 70, lamp 83, and filter 86 placed within container chamber 65 of container 60 with lid 61 enclosing container chamber. Lid 61 may then be positioned with respect to container 60 to allow a user to access container chamber 65. The user may then remove hood 20, duct 40, hood flange 27, flange 70, lamp 83, and filter 86 from container chamber 65. Following removal from container chamber 65, the user may position hood 20 from hood storage state 32 into hood deployed state 34, attached hood flange 27 to sheeting 29 of hood 20 (if necessary), position duct 40 from duct storage state 52 into duct deployed state 54, attach flange 70 to container 60, place filter 86 within hood flange passage 28 of hood flange 27, place lamp 83 within flange passage 75, and connect duct 40 to flange 70 and to hood flange 27. Lid 61 may then be sealingly secured to the remainder of container 60 thereby sealingly enclosing container chamber 65. Thus, hood 20, duct 40, container 60, hood flange 27, flange 70, lamp 83, and filter 86 are assembled into pathogen containment apparatus 10 in deployed state 14.

Following assembly of pathogen containment apparatus 10 into deployed state 14, hood 20 in hood deployed state 34 may be placed over the head and the upper torso of the patient to partially enclose the head and the upper torso. Fan unit 80 may then be activated by user input using user interface 94. When activated, fan unit 80 communicates air 99 into hood chamber 25 from ambient environment 98 through hood aperture 21 and from hood chamber 25 into container chamber 65 of container 60 via duct passage 45 of duct 40, and then discharges air 99 from container chamber 65 into the ambient environment 98 through vent 87. In accordance with negative pressure room laminar flow standards (OSHA/CDC), laminar flow of air 99 is created in a direction originating from the area of the patient's torso and flowing over the torso and head, in certain implementations. In various implementations, fan unit 80 may provide air flow sufficient for at least 12 changes per hour (ACH) of air 99 within hood chamber 25, which is at least the OSHA negative pressure room standard. In various implementations, fan unit 80 may provide air flow of at least 70 fpm face velocity, which is at least the OSHA vapor hood standard. Air 99 may entrain pathogens 96 from ambient environment 98 or from hood chamber 25. Pathogens 96 emitted from the patient within hood chamber 25 are then entrained in air 99.

Filter 86 in combination with lamp 83 eliminates pathogens 96 from air 99 so that air 99 generally devoid of pathogens 96 is discharged from container chamber 65 through vent 87 into ambient environment 98. Air 99 including pathogens 96 flows through filter 86 and filter 86 captures pathogens 96 thereby eliminating at least a portion of pathogens 96 from air 99 as air 99 is communicated along fluid pathway 15. As illustrated, some pathogens 96 may pass through filter 86. Lamp 83, which is downstream of filter 86, emits UV light 93 that eliminates any remaining pathogens 96 from air 99 by ionizing effects on pathogens 96. UV light 93 reflects off of material 84a, 84b, 84c thus permeating at least portions of duct passage 45, flange passage 75, and container chamber 65, respectively, ionizing pathogens 96, if any, within at least portions of duct passage 45, flange passage 75, and container chamber 65, in this implementation. Air 99 disinfected by filter 86 and UV light 93 is then discharged from container chamber 65 through vent 87 into the ambient environment 98, in this implementation. Accordingly, exemplary pathogen containment apparatus 10 eliminates pathogens 96 from air 99, so that air 99 generally devoid of pathogens 96 is discharged through vent 87 into the ambient environment 98. Thus, exemplary pathogen containment apparatus 10 eliminates pathogens 96 both from ambient environment 98 and pathogens 96 emitted by the patient.

Pathogen containment apparatus 10 may be disassembled from deployed state 14 into storage state 12 by removing duct 40 from hood flange 27 and flange 70, removing hood flange 27 from attachment to sheeting 29 (in implementations in which hood flange 27 is removable) removing flange 70 from container 60, removing filter 86 from hood flange passage 28 of hood flange 27, and removing lamp 83 from flange passage 75, positioning of hood 20 from hood deployed state 34 into hood storage state 32, and positioning duct 40 from duct deployed state 54 into duct storage state 52. Hood 20 in hood storage state 32, hood flange either attached to the sheeting 29 or removed from sheeting 29, duct 40 in duct storage state 52, flange 70, lamp 83, and filter 86 may then be placed within container chamber 65 of container 60 with lid 61 enclosing container chamber thereby facilitating transport and storage of pathogen containment apparatus 10. As pathogen containment apparatus 10 is disassembled from deployed state 14 to storage state 12, hood 20 including hood flange 27, duct 40, flange 70, container 60 including container chamber 65, filter 86, lamp 83, fan unit 80, and so forth may be cleaned and decontaminated. Filter 86 may be discarded and a new filter(s) 86 placed in container chamber 65. In certain implementations, various portions of pathogen containment apparatus 10 such as hood flange 27 may be colored a specified color (e.g., red) and labelled with text to indicate a requirement for cleaning and decontamination as pathogen containment apparatus 10 is disassembled from deployed state 14 into storage state 12.

Exemplary method 400 illustrates alteration of pathogen containment apparatus 10 from storage state 12 into deployed state 14 and alteration of pathogen containment apparatus 10 from deployed state 14 to storage state 12. Method 400 is entered at step 401 with pathogen containment apparatus 10 in storage state 12. At step 405, hood 20, duct 40, filter 86, lamp 83, and flange 70 are removed from container chamber 65 of container 60 following removal of lid 61 from sealing engagement with a remainder of container 60. After removal of hood 20, duct 40, filter 86, lamp 83, and flange 70, lid 61 is then sealingly secured to the remainder of container 60 thereby sealingly enclosing container chamber 65. Hood 20 is in hood storage state 32 and duct 40 is in duct storage state 52.

At step 410, hood 20 is altered from hood storage state 32 to hood deployed state 34. If necessary, hood flange 27 is attached to the remainder of hood 20, at step 410.

At step 415, duct 40 is altered from duct storage state 52 to duct deployed state 54.

At step 420, flange 70 is secured to container 60.

At step 425, filter 86 is positioned within hood flange passage 28 of hood flange 27.

At step 430, lamp 83 is positioned within flange passage 75 of flange 70.

At step 435, duct 40 is secured to hood flange 27 and to flange 70.

At step 440, hood 40 is positioned to partially enclose the head and the upper torso of the patient within hood chamber 25.

At step 445, air 99 is communicated along fluid pathway 15 as controlled by user inputs received from the user by user interface 94. Pathogen containment apparatus 10 is in deployed state 14 at steps 440, 445.

At step 450, duct 40 is removed from hood flange 27 and flange 40.

At step 455, lamp 83 is removed from flange passage 75.

At step 460, filter 86 is removed from hood flange passage 28.

At step 465, hood 20 is altered from hood deployed state 34 to hood storage state 32. Hood flange 27 may be removed from the remainder of hood 20, at step 465,

At step 470, duct 40 is altered from duct deployed state 54 to duct storage state 52.

At step 475, hood 20, duct 40, filter 86, lamp 83, and flange 70 are placed in container chamber 65. Container chamber 65 containing hood 20, duct 40, filter 86, lamp 83, and flange 70 is then enclosed by lid 61, at step 480. Pathogen containment apparatus 10 is in storage state 12 at step 480. As pathogen containment apparatus 10 is positioned from deployed state 14 to storage state 12 at steps 450 to 480, it should be noted that hood 20, duct 40, hood flange 27, flange 70, container 60 including container chamber 65, filter 86, lamp 83, and fan unit 80 may be decontaminated at steps 450 to 480, as appropriate, for example, by application of cleaning agent(s), and/or disinfectant thereto or in other ways, as would be readily recognized by those of ordinary skill in the art upon study of this disclosure. Filter 86 may be discarded as being contaminated. Thus, pathogen containment apparatus 10 in storage state 12 at step 480 is decontaminated and ready for redeployment by execution of steps 405 to 440. Method 400 terminates at step 491.

The foregoing discussion along with the Figures discloses and describes various exemplary implementations. These implementations are not meant to limit the scope of coverage, but, instead, to assist in understanding the context of the language used in this specification and in the claims. The Abstract is presented to meet requirements of 37 C.F.R. § 1.72(b) only. Accordingly, the Abstract is not intended to identify key elements of the apparatus and methods disclosed herein or to delineate the scope thereof. Upon study of this disclosure and the exemplary implementations herein, one of ordinary skill in the art may readily recognize that various changes, modifications and variations can be made thereto without departing from the spirit and scope of the inventions as defined in the following claims.

The invention claimed is:

1. A pathogen containment apparatus, comprising:
a container that defines a container chamber within;
a hood alterable between a hood storage position and a hood deployed position, the hood in the hood deployed position defines a hood chamber to partially enclose a head and upper torso of a patient;
a duct alterable between a duct storage position and a duct deployed position, the duct in the duct deployed position operably connectable to the hood in the hood deployed position and to the container to define a duct passage that forms at least a portion of a fluid pathway along which air is communicated into the hood chamber from an ambient environment, from the hood chamber to the container chamber, and from the container chamber through a vent to the ambient environment; and
wherein the hood in the hood storage position and the duct in the duct storage position are encloseable within the container chamber of the container.

2. The apparatus of claim 1, further comprising:
a fan unit disposable within the container chamber to communicate air via the fluid pathway.

3. The apparatus of claim 1, further comprising:
a filter positionable within the fluid pathway to remove pathogens from air communicated via the fluid pathway.

4. The apparatus of claim 1, further comprising:
a lamp positionable within the fluid pathway, the lamp emits an ultraviolet-c (UVC) wavelength light to disinfect air communicated via the fluid pathway.

5. The apparatus of claim 4, further comprising:
a reflective material that forms an interior surface enclosing portions of the fluid pathway to reflect the ultraviolet-c (UVC) wavelength light within the fluid pathway.

6. The apparatus of claim 1, wherein the hood is comprised of transparent plastic sheeting engaged with a plurality of struts rotatably positionable with respect to one another to allow the hood to be positioned between the hood storage position and the hood deployed position.

7. A pathogen containment apparatus, comprising:
a container that defines a container chamber within;
a hood positioned in a hood deployed position to define a hood chamber that partially encloses a head and upper torso of a patient, the hood alterable between the hood deployed position and a hood storage position;
a duct positioned in a duct deployed position and operably connected to the hood in the hood deployed position and to the container, the duct defines a duct passage that forms at least a portion of a fluid pathway along which air is communicated into the hood chamber from an ambient environment, from the hood chamber to the container chamber, and from the container chamber through a vent to the ambient environment; and
wherein the hood in the hood storage position and the duct in the duct storage position is enclosed within the container chamber of the container.

8. The apparatus of claim 7, further comprising:
a fan unit disposed within the container chamber to communicate air via the fluid pathway.

9. The apparatus of claim 7, further comprising:
a filter positioned within the fluid pathway to remove pathogens from air as air is communicated via the fluid pathway.

10. The apparatus of claim 7, further comprising:
a lamp positioned within the fluid pathway, the lamp emits an ultraviolet-c (UVC) wavelength light to disinfect the air as air is communicated via the fluid pathway.

11. The apparatus of claim 10, further comprising:
a reflective material that forms an interior surface of at least portions of the fluid pathway to reflect the ultraviolet-c (UVC) wavelength light within the fluid pathway.

12. The apparatus of claim 7, wherein the hood is comprised of transparent plastic sheeting engaged with a plurality of struts rotatably positionable with respect to one another to allow the hood to be positioned between the hood storage position and the hood deployed position.
